(19) United States
(12) Patent Application Publication
(10) Pub. No.: US 2024/0293227 A1
(43) Pub. Date: Sep. 5, 2024

(54) PROSTHETIC HEART VALVE COMPRISING A STENT STRUCTURE

(71) Applicant: BIOTRONIK AG, Buelach (CH)

(72) Inventor: Thomas Kuske, Rostock (DE)

(21) Appl. No.: 18/262,054

(22) PCT Filed: Feb. 15, 2022
(86) PCT No.: PCT/EP2022/053629

§ 371 Date: Jul. 19, 2023

(30) Foreign Application Priority Data
Feb. 16, 2021 (EP) 21157361.3

(51) Int. Cl. A61F 2/24

(52) U.S. Cl. CPC A61F 2/2418

(57) ABSTRACT

A prosthetic heart valve includes a stent structure configured to expand from a compressed state for transluminal delivery to a natural expanded state and having an overall height (H). The stent structure includes a mesh structure that has an essentially tubular shape and defines a circumference with contours, wherein the contours define a proximal inlet region and a distal outlet region. The proximal inlet region and the distal outlet region are directly connected to one another. The mesh structure includes a plurality of closed cells that in a longitudinal direction of the prosthetic heart valve have varying cell sizes and cell configurations to form a plurality of cell patterns that vary in size between the proximal inlet region and the distal outlet region. A valve is arranged inside a lumen of the stent structure. The overall height (H) of the stent structure is less than 45 mm.

### PRIORITY CLAIM

This application is a 35 U.S.C. 371 US National Phase and claims priority under 35 U.S.C. § 119. 35 U.S.C. 365(b) and all applicable statutes and treaties from prior PCT Application PCT/EP2022/053629, which was filed Feb. 15, 2022, which application claimed priority from European Application EP21157361.3 filed Feb. 16. 2021.

### FIELD OF THE INVENTION

The field of the invention is prosthetic heart valves, in particular stent-based prosthetic aortic valves.

### BACKGROUND

A heart valve operation is used to repair or replace diseased heart valves. A conventional heart valve operation involves a procedure conducted on the open heart and takes place under general anaesthesia. For this, in general an incision is made through the patient's sternum (so-called sternotomy), and the patient's heart function is stopped for the period of the intervention, blood being circulated using a heart-lung bypass machine during this period.

The conventional heart valve operation described above may be indicated if the natural heart valve narrows or is narrowed during the systole, which is generally called stenosis, or if the natural valve closes only incompletely during the diastole (insufficiency), so that there is a reverse flow into the ventricle. If the valve is replaced, the native valve is excised and replaced with a biological or mechanical valve.

Mechanical valves require anticoagulant medication for life to prevent the formation of blood clots. In addition, they are characterized by acoustic clicking by the artificial valve that can typically be heard through the chest cavity.

Biological tissue valves typically do not require such medication. Tissue valves may be obtained, for example, from human cadavers (homologous valve) or may be harvested from pigs or cows (xenogeneic heart valves); in addition, they are normally attached to artificial anchoring structures (e.g. a ring) that are then anchored to the patient's heart.

Conventional heart valve surgeries are highly invasive operations involving significant associated risks. Among these risks are hemorrhages, infections, stroke, cardiac infarction, arrhythmia, renal failure, side effects from the anesthesia, and sudden death. Two to five percent of patients die during the surgery.

After surgery, patients may be temporarily more or less seriously limited due to emboli and other factors associated with the heart-lung machine. The first two to three days following the operation are normally passed in an intensive care unit, where cardiac function can be closely monitored.

In order to address the aforementioned drawbacks, due to advances in minimally invasive surgery and in interventional cardiology, in the past 20 years researchers have been encouraged to pursue percutaneous aortic valve replacement.

For example, Percutaneous Valve Technologies (“PVT”), in Fort Lee, New Jersey, now Edwards Lifesciences, developed a balloon-expandable stent in which a bioprosthetic valve is integrated. This valve prosthesis is set in the region of the native valve, the native valve being pressed to the side by the stent and the artificial valve thus immediately assuming the valve function. In doing so, the stent, expanded by the balloon, anchors and seals the valve prosthesis. This device from PVT is designed to be implanted in a cardiac catheter laboratory under local anaesthesia and using fluoroscopic guidance, so that general anaesthesia and open-heart surgery can be avoided. Said device was implanted in a patient for the first time in April 2002.

The valve prosthesis from PVT has a number of technical drawbacks, however.

Use of the PVT stent is not reversible and the stent cannot be repositioned. This is a critical disadvantage, since incorrect positioning can block the patient's coronary arteries or lead to leaks in the valve all the way to complete migration of the valve.

Another drawback to the PVT device described in the foregoing is its relatively large cross-sectional profile. This valve prosthesis is mounted on a balloon catheter, which renders implantation through the aorta (trans-aortic implantation) difficult. Therefore, a transseptal approach, which requires puncturing the septum, may be necessary for this device in certain circumstances, and this significantly increases the risks of the procedure.

Other artificial replacement heart valves from the prior art use self-expanding stents as carrier structures for attaching, supporting, and anchoring the valve. In the case of endovascular aortic valve replacement procedures, precise setting of the aortic valve relative to the coronary arteries and mitral valve is critical. Standard self-expanding systems suffer from low implementation precision, however. The proximal end of the stent is frequently not appropriately released from the catheter system in the first approach, which renders precise and secure setting more difficult and may make necessary, for example, more intensive fluoroscopic exposure in order to be able to undertake precise repositioning. For the aforesaid reasons there are often discrepancies in where the precise inflow and outflow regions of the stent are disposed relative to the native valve, the coronary arteries, and the mitral valve.

Another drawback of previously known self-expanding heart valve systems from the prior art is the lack of radial stiffness and the radial force, which therefore does not build up enough, relative to the surrounding anatomy; this is particularly true in the annular region (Anulus aortae) of the native aortic valve and in the outflow region of the stent.

So that self-expanding stent systems may be advanced easily to the target location via a delivery catheter, it must be possible to compress (crimp) the metal towards the catheter diameter without the metal permanently deforming plastically and losing some of its desired and required radial force (so-called shape memory material—for example, nitinol). This must be assured, in particular, for multiple compressions (crimping), as normally occurs during repositioning of a valve prosthesis.

At present, known medically applicable alloys for self-expanding stents, such as nitinol, exhibit both lasting plastic deformation and a decrease in material strength (so-called cyclical fatigue) following multiple compressions (crimping), depending on the severity of the deformation. Both of these phenomena lead to a loss in the radial strength of the stent as a support structure for an artificial valve, for example an aortic valve, which then jeopardizes a secure seat for the stent in the surrounding anatomy. This may lead to undesired paravalvular leakage and thus to defective valvular function of the prosthetic heart valve.

If an artificial heart valve is attached in the stent, as is the case for aortic valve replacement (for example, in so-called TAVI or TAVR systems), the stent structure and thus also the valve attached thereto is significantly mechanically limited and challenged in the region of the vessel walls during the diastole. The force for holding back arterial pressure and preventing blood from returning to the ventricle during the diastole is transmitted directly onto the interface between stent and vascular wall. Therefore, the radial force required to keep the expanding stent for the artificial valve in contact with the vascular wall, and not to let it slip away, is much higher than for conventional stents (for example, stents for coronary blood vessels) that do not have valves in them.

Moreover, a self-expanding stent-without sufficient radial force-is limited in its function, is not tight against leaks, and may possibly migrate entirely.

U.S. patent application 2002/0151970, by Garrison et al., describes a two-part device for replacing the aortic valve and suitable for positioning through the aorta of a patient. According to this application, a stent is percutaneously placed over the native aortic valve, and then an artificial valve is positioned in the lumen of the stent. Due to this separation of the stent and valve portion during positioning, the profile of the catheter for the device may be reduced enough to permit positioning via the aorta (trans-aortic positioning) without requiring transseptal access. Both the stent and the artificial valve may be balloon-expandable or self-expanding.

The devices described in the Garrison patent application do use a trans-aortic approach for positioning, but nevertheless suffer from a number of drawbacks.

Initially only the stent part of the device is implanted in a single step and as a single piece in the region of the native valve. Since the valve structure is not implanted into the already set stent until a later step, positioning of the stent—without valve—cannot be functionally evaluated. Any initial incorrect positioning or undesired shortening or migration of the stent during its expansion may lead to incorrect final alignment of the entire valve device.

Other drawbacks of the previously known stent structure, in particular, stent structures for aortic valve prostheses, may be summarized as follows:

It is known that excessive radial forces in a stent structure can cause conduction problems that may themselves make necessary additional use of a cardiac pacemaker. On the other hand, in conventional stent structures, the radial forces decrease sharply following multiple repositionings and are thus not adequate for sealing the stent structure against the surrounding anatomy. To counteract this, stent structures from the prior art use external tissue for additional sealing for valve prostheses. However, this leads to larger crimp profiles for the valve prostheses. For this reason, such stent structures must permit smaller crimp diameters, which to date does not seem to have been adequately accomplished. One solution in this regard is a 12 to 6-cell division of the proximal inlet region (12) compared to the distal outlet region (6) of the stent. The greater netting of the cells in the inlet region, with 12 cells, still does not appear to be sufficient for permitting smaller crimping diameters and simultaneously building up adequate radial force for sealing against the anatomy.

With respect to the aforesaid examples of drawbacks of the prior art that are associated with previously known techniques for percutaneous replacement of a heart valve, it is desirable to provide vascular implant devices, in particular a stent-based prosthetic heart valve, that overcome these drawbacks.

More recently, minimally invasive systems and techniques have been developed to facilitate catheter-supported implantation of a valve prosthesis in the beating heart and thus avoid the need for a classical sternotomy and cardiopulmonary bypass. With transcatheter (or transluminal) techniques, a heart valve prosthesis is sealed in a catheter for insertion and then advanced to the heart, e.g. through an opening in the femoral artery, subclavian artery, aorta, or ventricular apex, in order to gain access to the aortic valve in this manner. The prosthesis delivered is then set in the so-called “aortic annulus” of the valve to be replaced.

The heart valve prosthesis that is generally used in transcatheter methods includes an expandable, multi-stage frame or stent that supports a valve body having two or more valve leaflets. The actual shape and configuration of a specific prosthetic heart valve is partly a function of the native shape and size of the valve to be repaired (for example, aortic valve, mitral valve, tricuspid valve, or pulmonary valve).

In general, prosthetic heart valves seek to replicate the functions of the valve to be replaced, and the stent used with the prosthesis determines the final size and shape of the valve. Moreover, the stent anchors the transcatheter valve prosthesis in or about the native annulus. One type of transcatheter valve stent frame may initially be provided in an expanded or non-curved state, then may be pressed or compressed about a balloon segment of a catheter. The balloon is then inflated in order to expand and set the prosthetic heart valve. In other supported prosthetic heart valve designs, the stent frame is shaped such that it expands automatically. In these systems, the valve stent is crimped to a desired size and kept in a sleeve in this compressed state for transluminal delivery. Retracting the sleeve from this valve stent permits the stent to expand automatically to a larger diameter and fix at the native valve location. As a rule, conventional suturing of the prosthetic heart valve to the patient's native tissue is not required in either of these types of devices for administering percutaneously compressed heart valves.

In order to achieve long-term anchoring to the native valve location, the stent frame must have and maintain increased strength and resistance to radial forces or pressures. A prosthetic valve that is not anchored to adequately withstand the forces of the continuously varying vascular wall diameter and turbulent circulation there can detach or become ineffective in some other manner (as already described in the foregoing). Moreover, it is desirable to select the size or length of the stent such that increased interaction with the native anatomy is assured. Mesh-like stent structures, for example made of nitinol, have proved quite suitable for satisfying these requirements, and are conventionally configured such that they have a repeating pattern of tightly dimensioned, shaped, and arranged cells. However, it has been found that, following implantation, previous stent-based valve prostheses may lead to the further need for cardiac pacemakers-simply due to issues related to conduction; see above.

Given this background, there is a need for optimizing stent-based prosthetic heart valves that can offer sufficient and secure anchoring in the surrounding anatomy and do not have any effect, or have only a minor effect, on the conducting paths of the heart.

Document EP 3 184 082 A1 describes a stent for a surgical valve that forms three interconnected sections, namely a proximal inflow section, a middle section and a distal outflow section. In order to provide relief from tricuspid valve leakage or regurgitation and from mitral valve leakage or regurgitation as well as to minimize the recovery time of a patient following the treatment with such valve, a maximum outer diameter of the middle section is larger than a maximum outer diameter of the proximal section, wherein the proximal section has an at least substantially equal outer diameter over its length. However, the stent has a quite complicated outer form with a comparable long structure. The structure makes it more difficult for the physician to correctly place the stent within the natural valve so that the implant has the required flexibility and optimum radial force distribution.

As described in the foregoing, minimally invasive aortic valve replacement is generally used today, even for treating acquired aortic valve stenoses caused by local calcifications. For these types of stenoses, the atrioventricular valves and annulus of a heart are normally more or less highly calcified. These calcifications take the shape of deposits of different hardness that induce geometric changes in the normal anatomy and thus also limit the natural deformability of the tissue structures.

For self-expanding stents that are used as supports and for anchoring a TAVI or TAVR prosthesis, a number of technical properties are essential to ensure post-procedural clinical success.

These properties include:
sufficient radial force for the stent structure for secure anchoring, but without precipitating conduction problems, in particular following multiple repositionings of a TAVI valve or TAVR valve;
free access to the coronary arteries;
stent structure buckling resistance when the TAVI valve or TAVR valve is repositioned and in severely deformed annuli; and,
sufficient sealing tightness for the TAVI valve or TAVR valve.

Another requirement for the functioning of a TAVI valve or TAVR valve is a corresponding outer shape of the implantable prosthesis, the shape fitting the anatomy in question and thus having the required flexibility. In addition, it is advantageous when the geometry of the valve itself does not change at all, or changes only minimally as a function of the available annulus diameter.

One critical property of a TAVI stent or TAVR stent is thus providing an optimum radial force and thus optimum distribution of the radial force longitudinally and around the circumference of the stent structure. The radial force must be of a certain magnitude in order to securely anchor the stent and to assure sealing tightness with respect to the anatomy. On the other hand, the radial force must not be too great, either, because otherwise the heart's conduction system is irritated or it is even possible for tissue damage to occur (see above).

Adequate radial force still must always be assured, in particular, following multiple repositionings of the stent structure, in which the latter is re-sheathed in a catheter capsule each time. However, nitinol, as a classic stent material, has a cyclical instability that leads to a decrease in the diameter of the stent, and thus to a reduction in the radial force, following each repositioning. Since this effect is a function of the extent of the elongations and the size of the affected region, it is desirable to provide a stent structure, as support structure for a prosthetic heart valve, that minimizes the elongations occurring and maintains an optimum radial force, distributed optimally across the stent structure itself.

Furthermore, even following TAVI implantation or TAVR implantation, the coronary arteries must remain accessible for further interventions by a catheter. This applies, in particular, following a reintervention in which a second prosthesis is set into a prosthesis that has already been implanted (so-called valve-in-valve principle).

In addition, the geometric dimensions of the valve, in particular diameter and height, for optimum functioning as valve (characterized inter alia by complete valve opening, reliable closing of the valve, pressure loss, coaptation, and especially adequate service life) are essential. Therefore, a stent design is to seek to ensure that the geometry of the valve remains the same, to the greatest possible extent, across the permissible diameter range for the annulus. The change in the geometry of the valve between completely expanded stent and implanted configuration should also be as small as possible. Further, less material usage is desirable also for lesser material's load of the patient.

From the literature, it is also known that stent-like structures can collapse inward, either when being re-sheathed in a catheter capsule or when implanted in highly calcified annuli (so-called “buckling” or “infolding”). This buckling or infolding is generally caused by mechanical instability of the stent structure. The prosthesis loses some of its functionality until it fails completely.

For the physiological functioning of a valve prosthesis, also the total prosthesis height must be considered; e.g. in view of the transition of the sinus into the ascending aorta and free access to the right and left coronary ostium. For instance, a certain minimum length must be provided, so that when the prosthesis is repositioned the stent structure is still fixed distally in the catheter capsule on the one side and the prosthesis can deploy proximally such that the valve is already functioning in order to be able to evaluate the functioning of the entire prosthesis prior to complete release.

### SUMMARY OF THE INVENTION

A preferred prosthetic heart valve includes a stent structure configured to expand from a compressed state for transluminal delivery to a natural expanded state and having an overall height (H). The stent structure includes a mesh structure that has an essentially tubular shape and defines a circumference with contours, wherein the contours define a proximal inlet region and a distal outlet region. The proximal inlet region and the distal outlet region are directly connected to one another. The mesh structure includes a plurality of closed cells that in a longitudinal direction of the prosthetic heart valve have varying cell sizes and cell configurations to form a plurality of cell patterns that vary in size between the proximal inlet region and the distal outlet region. A valve is arranged inside a lumen of the stent structure. The overall height (H) of the stent structure is less than 45 mm.

### BRIEF DESCRIPTION OF THE DRAWINGS

FIG. 1 shows outer contours of one embodiment of the prosthetic heart valve of the present invention;

FIG. 2 is a stent structure in a side view according to one embodiment;

FIG. 3 is a side view of a stent structure according to a second embodiment of the prosthetic heart valve of the present invention;

FIG. 4 shows a perspective view of the stent structure of FIG. 3 from the side;

FIGS. 5A and 5B show crimped states of a preferred stent structure;

FIGS. 6A and 6B show crimped states of preferred stent structure;

FIG. 7 is a side view of a stent structure according to a third embodiment of the prosthetic heart valve of the present invention;

FIG. 8 shows a perspective view of the stent structure of FIG. 7 from the side;

FIG. 9 is another side view of the stent structure of FIG. 7;

FIG. 10 shows an alternative arrangement of struts in the outflow region for the stent structure of FIG. 7;

FIG. 11 shows an exemplary outer shape of the stent structure of the present invention according to. FIG. 1 in the expanded state having exemplary dimensions and angles in the context of the invention [in mm];

FIG. 12 shows another exemplary outer shape of the stent structure of the present invention according to. 1 in the expanded state having exemplary dimensions and angles in the context of the invention [in mm];

FIGS. 13A and 13B show shifted connectors of the invention of FIG. 2 with example corresponding dimensions [in mm] and angles;

FIG. 14 shows shifted connectors of the invention of FIG. 2 with example corresponding dimensions [in mm] and angles;

FIG. 15 shows a stent structure in accordance with FIG. 2 and a strut width that vary along the struts;

FIG. 16 shows commissure posts for attachment of a valve arrangement to the stent structure of the invention;

FIG. 17 shows commissure posts for attachment of a valve arrangement to the stent structure of the invention;

FIG. 18 shows commissure posts for attachment of a valve arrangement to the stent structure of the invention;

FIG. 19 shows commissure posts for attachment of a valve arrangement to the stent structure of the invention;

FIGS. 20A and 20B show a step of the manufacturing method of the stent structure of the FIG. 2;

FIG. 21 shows a step of the manufacturing method of the stent structure of the FIG. 2;

### DETAILED DESCRIPTION OF THE PREFERRED EMBODIMENTS

Preferred embodiments of the invention provide a stent-based prosthetic heart valve, that provides;
a) a reliable sealing tightness of the stent structure of the present invention against the surrounding anatomy, such as, for example, the annulus region of an aortic valve, is assured by a finely netted inlet region that, via its strut and cell design, builds sufficiently high radial force for sealing, and this is even true following multiple repositionings, if any, and with a sufficiently small crimp diameter that permits the use of an additional sealing tissue exteriorly on the stent structure in the inlet region (for example, of an outer skirt-shaped element);
b) the geometry of the valve arrangement sutured into the stent structure changes only minimally due to a specially configured outer shape of the stent structure if the diameter of the inlet changes according to the annulus diameter; correspondingly, the geometry of the valve arrangement of the present invention is independent, to the greatest degree possible, of changeable annular diameters;
c) the stent structure is configured in a stable manner such that buckling is prevented in the longitudinal direction of the stent, both when the stent is re-sheathed in a catheter capsule and given severely deformed annuli with significant calcifications;
d) the stent structure provides a smaller overall crimp profile leading to smaller foreshortening when elastic recoil occurs; and
e) the stent structure is having an overall stent height that is reduced to a physiologically acceptable minimum, and thus without affecting the prosthetic heart valve's performance.

Furthermore, the specially configured outer shape of the stent structure of the present invention permits improved hemodynamics despite intentionally increased radial force distribution.

A preferred prosthetic heart valve includes a stent structure and a valve arrangement. The valve arrangement is arranged inside a lumen of the stent structure. The stent structure is configured such that it may automatically expand from a compressed state for transluminal delivery to a natural, expanded state (self-expanding stent structure).

The prosthetic heart valve of the present invention includes a stent structure that is configured to expand from a compressed state for transluminal delivery to a natural expanded state and having a minimized overall height, the stent structure including a mesh structure that has an essentially tubular shape and that furthermore defines a circumference with contours, wherein the contours define a proximal inlet region and a distal outlet region, wherein the inlet region and outlet region are directly connected to one another, and wherein the mesh structure includes a plurality of closed cells that in a longitudinal direction of the prosthetic heart valve have varying cell sizes and cell configurations, and thus includes a plurality of cell patterns that vary in size between the proximal inlet region and the distal outlet region, and a valve arrangement that is arranged inside a lumen of the stent structure, wherein the overall height of the stent structure (including the connectors) is low, preferably less than 45 mm, further preferably less than 40 mm, more preferably less than 35 mm.

Hence, the above heart valve prosthesis includes a short stent structure which improves coronary access, eases “valve in valve” implantation, if indicated, prevents or minimizes contact between stent and aorta wall because it has a smaller capsule height in the natural expanded state compared with conventional stent structures for a prosthetic heart valve.

The height is the dimension of the stent structure in longitudinal direction (i.e. the direction of the longitudinal axis of the stent structure-an essentially tubular shape).

Another advantage is that less material is necessary for stent structure production and less material is implanted into the patient's body leading to a lower material's load of the patient. Additionally, the shortened stent design of the invention is less prone to forming imprints in the valve arrangement in the compressed state during implantation. The inventive prosthetic heart valve avoids a tilting of the stent structure during positioning and re-positioning as well as avoids buckling. It further provides an improved fixation in the annulus plane.

Each of the closed cells includes a plurality of struts that are connected to one another.

The reduced outlet diameter that prevents complete circumferential or partial circumferential contact between stent outlet and vascular wall of the ascending aorta; thus, the outlet region of the present invention and the outlet of the configuration has reduced sealing, anchoring, or aligning function in the stent structure.

In one embodiment, the cells in the inlet region are formed and arranged for defining in the stent structure
a conical-convex outer shape across the entire circumference of the inlet region (i.e. a so-called “conical-convex inlet region” or “conical-convex inflow region”), or
a linear cylindrical outer shape across the entire circumference of the inlet region (i.e. a so-called “linear cylindrical inlet region” or “linear cylindrical inflow region”), and the cells in the outlet region are shaped and arranged for defining in the stent structure a linear cylindrical outer shape across the entire circumference of the outlet region (i.e. a so-called “linear cylindrical outlet region” or “linear cylindrical outflow region”). This stent structure results in a smaller overall crimp profile which reduces foreshortening when arranged in a delivery device.

In one embodiment, the cells of the inlet region and outlet region are configured differently from one another to always build up, when the stent structure is in the expanded state, a higher maximum radial force in the inlet region in direct comparison to the lower maximum radial force in the outlet region. With this design, the prosthetic heart valve may be set relative to the native anatomy such that the inlet region builds a radial force intentionally elevated compared to the radial force of the outlet region and transition region in order to securely anchor itself in the annulus region, but at the same time the radial force of the inlet region is not increased such that it has a negative effect on the heart's conduction paths or impedes conduction.

In some embodiments, a dimension of at least one of the struts of the closed cells in the inlet region is smaller than a corresponding dimension of a corresponding strut of the closed cells in the outlet region. In other embodiments, the inlet region and the outlet region include a node element that connects two struts of each of the closed cells to one another, and one dimension of at least one of the node elements in the inlet region is smaller than a corresponding dimension of each of the node elements in the outlet region.

In this context, the cells in the inlet region are configured to have the highest radial force along the circumference of the inlet region of the stent structure in its natural state than the corresponding cells in the outlet and transition regions.

Still other aspects according to the principles of the present invention relate to a method for treating a patient's native heart valve, for example a patient's aortic valve. The method includes supplying a prosthetic heart valve of the present invention as described herein to the native heart valve, for example to a patient's native aortic valve. In this case, the step for delivering the prosthetic heart valve includes retaining the stent structure in the compressed state inside a delivery device. The prosthetic heart valve is then set by the delivery device, including the stent structure, which expands in the direction of the natural state, into the native heart valve. The inlet region (having the highest radial force) is oriented to a desired anatomical position of the native heart valve, for example the annulus of an aortic valve. In some embodiments, therefore, the native heart valve is an aortic valve, and the desired anatomical position is disposed in the annulus.

The aforesaid object of the invention is attained using the prosthetic heart valve of the present invention, in particular using the included stent structure, as follows:

The present invention relates to vascular implant devices. In particular, the present invention refers to stent-based vascular implants, preferably including an artificial heart valve for endovascular or percutaneous replacement of a native heart valve. The invention is directed in particular to an aortic valve prosthesis (TAVI valve or TAVR valve) that can replace a patient's natural aortic valve.

Sufficient sealing of the prosthetic heart valve is also be provided by cells in the inlet region of the stent that are relatively small compared to the rest of the stent, since small cells are better able to adapt to anatomical irregularities in the annulus region, for instance if there are calcifications.

According to the invention, the stent should therefore have larger cells in the outlet region than in the inlet region, the inlet region and outlet region being directly connected in order to reduce the overall height of the stent structure. Accordingly, there is no transition zone as in prior art stent structures. The diameter of the outlet region should be selected such that no functionally relevant contact occurs between the wall of the aorta and the outlet region of the stent structure. That is, the outlet region of the stent structure should be designed to produce no complete circumferential contact to the surrounding anatomy or to produce only partial circumferential contact to the surrounding anatomy, such contact resulting, however, in no functionally relevant contact for the prosthetic valve prosthesis.

The design-dependent shortening of the stent structure during the expansion should therefore be intentionally used for active foreshortening of the stent to the anatomically reasonable length following complete implantation.

In one embodiment, the prosthetic heart valve of the present invention includes a stent structure as a support structure and a valve arrangement for unidirectional valve function (inlet and outlet direction are oriented only in one direction). In one embodiment of the prosthetic heart valve of the present invention, the valve arrangement is an artificial replacement valve that is attached to the stent structure, preferably an artificial TAVI valve or TAVR valve including a plurality of valve leaflets, more preferably three valve leaflets, and a plurality of skirts, more preferably a plurality of skirts within the stent structure (inner skirt) and one or more skirts outside of the stent structure (outer skirt). Even more preferably, three inner skirts are attached to the stent structure and one or more outer skirts are attached to the outside. The stent structure includes a self-expanding stent, preferably a self-expanding stent structure including nitinol.

The valve arrangement (the replacement valve), in particular the TAVI valve or TAVR valve, for example including a plurality of valve leaflets that are arranged for defining an entry side and an exit side in the prosthetic heart valve, wherein the valve arrangement is designed to be attached inside the stent structure using sutures or adhesives, preferably using sutures at suitable positions on the stent structure, and as such to be released endovascularly in the native aortic region of the patient's heart in order to replace the patient's native aortic valve.

In one embodiment the valve arrangement may have two or more valve leaflets, for example three valve leaflets, and includes an inner skirt element on the inside of the stent structure, wherein said valve leaflets are sutured or glued at least to the inner skirt element and the inner skirt element is itself sutured or glued to the stent structure.

In one embodiment of the invention, the expandable stent structure is produced from a single metal strand, preferably nitinol, MP35N or L605.

In one embodiment of the invention, the expandable stent structure includes a convex inflow region.

In one embodiment of the invention, the expandable stent structure includes a conical-convex inflow region or a linear cylindrical inflow region.

In one embodiment of the invention, the expandable stent structure includes a conical-convex inflow region that extends proximally toward the inlet of the stent structure or a linear cylindrical inflow region having a constant diameter.

In one embodiment of the invention, the expandable stent structure includes a linear cylindrical outflow region.

In one embodiment of the invention, the expandable stent structure includes a linear cylindrical outflow region having a constant diameter.

In one embodiment of the invention, the expandable stent structure includes one or more connector elements at the distal end of the linear cylindrical outflow region/at the distal outlet region. The connector elements are used for a better connection of the prosthetic heart valve to a catheter during an implantation into a patient.

In one embodiment of the invention, the expandable stent structure includes one or more connector elements at the distal end of the linear cylindrical outflow region, the aforesaid connector elements having an eyelet.

At the very distal end of the distal outlet region two struts of a cell form an apical node (i.e. an apex or tip). The stent structure has (apical) nodes at its distal outlet region. The (apical) nodes are nodes of struts being located at the very distal end of the stent structure at the distal outlet region.

In one embodiment the distal outlet region includes at least one first connector element and at least one second connector element, the first connector element is connected to a first (apical) node of the stent structure via a first strut and the second connector element is connected to a second (apical) node of the stent structure via a second strut, wherein the first strut has a different length than the second strut. The first and second connector element each may include an eyelet. Due to the different length of the first and second strut the connector elements are shifted against each other so that the diameter of the stent structure in the crimped state is less than if the first and second strut would have the same length. This enables obtaining a stent structure having a low crimping diameter. Preferably, the first strut has a length which is equal or higher than the sum of the length of the second strut and the length of the second connector element. For example, the first strut has a length which is equal or higher than the sum of the length of the second strut and the outer diameter of the eyelet of the second connector element.

In one embodiment of the invention, the outlet region includes at least one first connector element and one second connector element at its distal end, each of the first connector element and the second connector element is connected to a respective apical node, wherein a first strut connecting the first connector element to a first node has a different length compared to a second strut connecting the second connector element to a second node, wherein the first node and the second node are located adjacent along the circumferential direction of the stent structure. In this embodiment, the connector elements are shifted against each other so that the diameter of the stent structure in the crimped state is less since one connector element with the shorter strut is located adjacent the longer strut of the adjacent connector element and vice versa.

In one embodiment, the first strut is longer than the second strut, for example longer than the length of the second strut and the radius of the second connector element.

In another embodiment of the invention, the aforesaid connector elements have atraumatic apical structures in the distal direction.

In one embodiment the stent structure has six closed cells in the distal outlet region and fifteen closed cells in the proximal inlet region, wherein the closed cells in the distal outlet region are larger than the cells in the proximal inlet region. At the very distal end of the distal outlet region two struts of the closed cell form an (apical) node. Thus, the stent structure has six (apical) nodes at its distal outlet region. Each of the six (apical) nodes includes a connector element having an eyelet. The distal outlet region includes three first connector elements and three second connector element, wherein each first connector element is connected to a first (apical) node via a first strut and each second connector element is connected to a second (apical) node via a second strut, wherein the first strut has a different length than the second strut. This enables a space saving crimping of the prosthetic heart valve. The stent structure has three commissure posts to which the three valve leaflets can be attached.

In one embodiment of the invention, the expandable stent structure includes a conical-convex inflow region or a linear cylindrical inflow region as described in the foregoing that expands proximally toward the inlet, a linear cylindrical outflow region as described in the foregoing including one or more connector elements at the distal end of the outflow region

In one embodiment of the invention, the expandable stent structure includes a conical-convex inflow region as described in the foregoing that expands proximally toward the inlet, a linear cylindrical outflow region as described in the foregoing including one or more, for example six, distal connector elements.

In one embodiment of the invention, the strut width of individual struts or of entire zig-zag rows of the stent structure may vary across the longitudinal (axial) length.

In one embodiment the strut width is greater than 0.25 mm. In one embodiment the main strut width is greater than 0.35 mm. In one embodiment the wall thickness is greater than 0.46 mm, preferably equal to or greater than 0.5 mm.

In another embodiment of the invention, the strut width of the struts of one or a plurality of zig-zag rows may be wider in the centre between two nodes than in the immediate vicinity of the nodes (so-called belly-configuration).

In another embodiment of the invention, the strut width of the struts of one or a plurality of zig-zag rows may be thinner in the centre between two nodes than in the immediate vicinity of the nodes (so-called waist-configuration).

In another embodiment of the invention, individual zig-zag rows or all zig-zag rows have a uniform strut length.

In another embodiment of the invention, individual zig-zag rows or all zig-zag rows have a non-uniform strut length.

In one embodiment of the invention, the cells in the transition region have the largest cell surface area of the entire stent structure of the present invention, in particular in the region of the coronary arteries.

The stent structure and the valve arrangement (for example, a TAVI valve or a TAVR valve) of the prosthetic heart valve for the present invention are preferably configured for endovascular positioning and deployment, preferably for transfemoral and/or transaortic positioning and deployment.

The stent structure of the present invention is furthermore suitable for secure and reliable fixation using the conical-convex inflow region at the height of the native aorta annulus of the native aortic valve.

In one embodiment of the invention, the stent structure is designed in the conical-convex inflow region such that the stent structure, regardless of the current annulus diameter, does not significantly affect the valve geometry or function and has radial force that is sufficient for fixing the stent in this region.

In one embodiment of the invention, in its expanded state the stent structure includes a conical-convex inflow region having a first diameter and a linear cylindrical outlet region having a second diameter, the first diameter of the inflow region being larger than the second diameter.

In one embodiment of the invention, the prosthetic heart valve includes a stent structure that has a rest configuration, the stent structure including a shape-memory material, preferably nitinol, that in the rest configuration is thermoset.

In one embodiment of the invention, the stent structure of the prosthetic heart valve has an intentionally configured mesh structure that leads to controlled shortening of the stent structure during endovascular implantation.

In one embodiment of the invention, the stent structure of the prosthetic heart valve has an intentionally configured mesh structure in the conical-convex inflow region that leads to controlled shortening of the stent structure in the inflow region during endovascular implantation.

In one embodiment of the invention, the stent structure of the prosthetic heart valve has an intentionally configured mesh structure in the linear cylindrical outflow region that leads to controlled shortening of the stent structure in the outflow region during endovascular implantation.

In each of the embodiments of the invention described herein, the mesh structure of the stent structure may be adapted for a controlled foreshortening of the stent structure during implantation.

The valve arrangement of the present invention (for example, a TAVI valve or TAVR valve) of the stent structure of the present invention is disposed inside the stent structure and is designed to admit a blood flow during the systole and to prevent blood from flowing backward during the diastole following deployment (unidirectional blood flow).

In one embodiment of the invention, at least part of the stent structure is covered by a biocompatible film or pericardium, and possibly by an additional element on the inside of the stent structure, which element is configured for reducing paravalvular leakage and regurgitation.

In one embodiment of the invention, at least part of the stent structure is covered by a biocompatible film or pericardium and possibly by an additional element that is configured for reducing paravalvular leakage and regurgitation.

In one embodiment of the invention, at least part of the stent structure is covered by a biocompatible film or pericardium, and possibly by an additional element on the inside of the stent structure, and at least in part is covered by a biocompatible film or pericardium and possibly an additional element on the outside of the stent structure, which element is configured for reducing paravalvular leakage and regurgitation.

Another aspect of the invention provides a prosthetic heart valve of the present invention for endovascular replacement of a patient's heart valve including: a customized stent structure according to the invention, and a suitable valve arrangement that fits therewith (for example, a TAVI valve or TAVR valve), wherein the customized stent structure is embodied to form, with the valve arrangement, a composite device that replaces the natural heart valve in an endovascular manner.

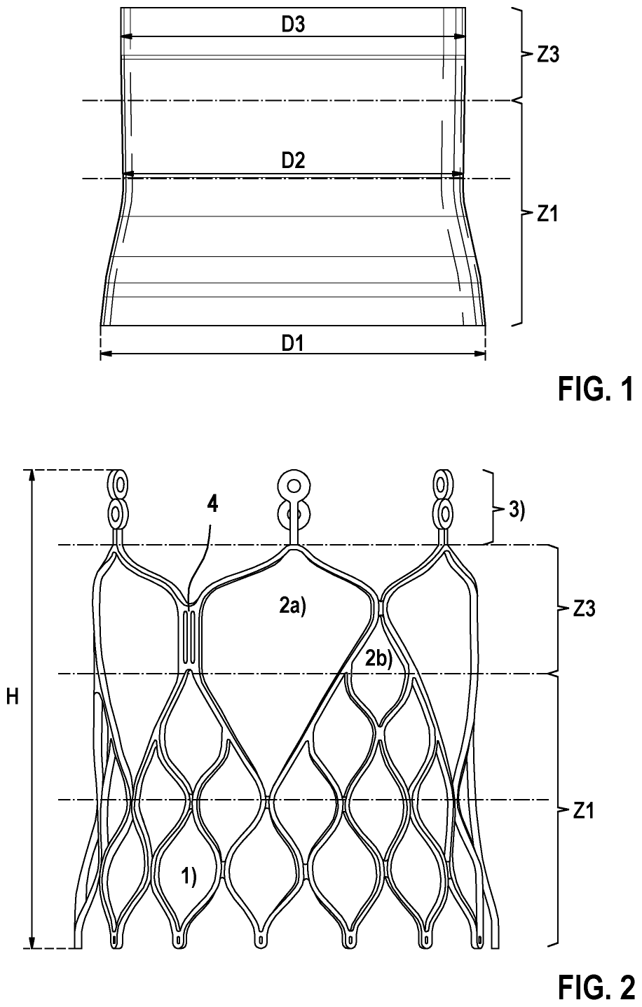

With respect to FIGS. 1 through 21, the present invention includes the following additional embodiments:

In one embodiment of the invention, the stent structure has a substantially conical-convex inflow region (Z1) (so-called annulus zone) that is defined by a first diameter (D1) and a second diameter (D2; “Belly Nadir”), D1 being larger than D2 and the lower outer surface area of this Z1 region being characterized in that it is curved outward in the longitudinal direction (convex or double curved).

In one embodiment of the invention, the zone Z1 connects to a linear cylindrical outflow zone (Z3) (so-called outflow region) having a second diameter (D3; so-called “Attachment” diameter). The diameter D3 in the entire zone Z3 is fixed and thus remains the same. This leads to a so-called “linear cylindrical outflow” of the prosthetic heart valve in the present invention.

In one embodiment of the invention, the aforesaid diameter D2 is always smaller than the diameter D3.

In one alternative embodiment, the aforesaid diameter D2 is equal to diameter D3.

With the above context, in another embodiment of the invention the expression “linear” is to be understood as “substantially linear” by the person skilled in the art, meaning that slight deviations in diameter along the outflow zone Z3 may occur; however, still resulting in a substantially linear outer shape of said zone Z3.

Another embodiment of the invention is characterized by a stent structure that has a certain number of cells in the circumferential direction in the inflow region (Z1), wherein said number of cells is divisible by 3 in order to provide a ⅓ symmetry; furthermore characterized in that another number of cells in the circumferential direction are present in the outflow region (Z3) and is also divisible by 3, but this number is less than the number of cells in the aforesaid inflow region (Z1).

In one embodiment, the number of cells in the inflow region is equal to the number of cells in the outflow region, but, for example, both number of cells is divisible by 3.

In one embodiment of the invention, the conical-convex inflow region (Z1) has 12, 15 or 18 cells (1), wherein the cells of one row may, for example, be formed as a rhombohedral cells, honey-comb shaped cells, V-shaped cells or N-shaped cells. In one embodiment, the linear cylindrical outflow region (Z3) has of one row of closed cells including 3 to 9 cells (2). In this configuration, the connection between inflow region and outflow region may be produced, for example, by 3 attaching elements (4).

In one embodiment, the distal end of the inflow region (Z1) may be partially formed by greater cells extending into the outflow region (Z3), wherein the greater cells alternate with smaller cells in the circumferential direction.

In the context of the invention, the stent structure having an inflow region and outflow region as described in the foregoing may furthermore be designed such that the foreshortening of the inflow region and outflow region may be influenced independently of one another using the number and/or specific embodiment of the stent element, that is, may be controlled in an intentional manner, so that the length of the stent in these regions may be adjusted during and following implantation such that a desired “foreshortening compensation” is induced in these regions, independently of one another, and/or so that undesired “foreshortening compensation” is minimized.

Correspondingly, in one embodiment of the invention, the stent structure described in the foregoing is provided with a defined and intentionally induced “foreshortening compensation” that may be used to ensure sufficient stent length during release from a catheter shaft so that valve function may be started early during the implantation, and after implantation the stent may be intentionally shortened in order to attain an anatomical fit in the native valve region.

In one embodiment of the invention, the stent structure described in the foregoing is furthermore characterized in that 3 to 4 closed zig-zag rows in the inflow region are provided, for example with a strut length adjusted to the entry diameter, wherein the length of the first two zig-zag rows (meander), i.e. its dimension in longitudinal direction, is greater than the length of the third and, if applicable, fourth zig-zag rows.

In the context of the present invention, the stent structure described herein is designed such that the struts of the stent are arranged such that the stent structure may be completely re-sheathed in a release capsule of a catheter multiple times. A preferred number of re-sheathings is at least three times, more preferably three times.

In one embodiment of the invention, the struts are characterized in that they have a strut width that may vary along the struts so that the width in the centre of the struts is smaller than at the node elements and each strut nevertheless has the same length (so-called waist-configuration).

In one embodiment of the invention, the struts are characterized in that they have a strut width that may vary along the struts so that the width in the centre is smaller than at the node elements and each strut nevertheless has the same length, but they are shorter than the length of the struts.

In one alternative embodiment, the struts are characterized in that they have a strut width that may vary along the struts so that the width in the centre is greater than at the node elements and each strut nevertheless has the same length (so-called belly-configuration).

In one alternative embodiment, the struts are characterized in that they have a strut width that may vary along the struts so that the width in the centre is greater than at the node elements and each strut nevertheless has the same length, but they are shorter than the length of the struts.

In one embodiment of the invention, the stent structure described in the foregoing is characterized in that the struts have a strut width that may vary along the struts so that the width is minimal at two positions along the strut and is the same in a preferred configuration. The strut width between the two positions is larger but equal to or smaller than the strut width at the nodes (so-called “double waist”).

The stent structure of the present invention has a plurality of technical advantages over the prior art:

The stent structure according to the embodiments has sufficient and relatively stable radial force that is always highest in the inflow region so that multiple repositionings, preferably three repositionings,—for example of a TAVI valve or TAVR valve based on this stent structure—are possible and the radial force remaining after the repositioning, particularly in the inflow region, is higher than the remaining radial force of comparable valve prostheses from the prior art. At the same time, unimpeded access to the coronary arteries is provided due to the configuration of the stent structure including a finely netted inlet region (corresponds to higher number of cells compared to the outlet region) and a coarse mesh outlet region (corresponds to lower number of cells compared to the inlet region).

Simultaneously, reliable sealing of the prosthesis is attained by using smaller, more finely netted cells in the inlet region, in particular due to the build-up of the highest radial force in the conical-convex inflow region compared to the other parts of the mesh structure of the stent of the present invention.

The ability of the coronary arteries to be reached, mentioned in the foregoing, is even further enhanced by the present design of a linear cylindrical outlet region of the stent structure, since the dimensions of the outlet region of the present invention are selected to not produce any further contact between stent structure and patient's vascular wall. The delivery catheter may thus pass unimpeded.

Due to the high variability in the anatomy of the ascending aorta, when the anatomy is not favourable, such as, for example, if there is an early and sharply infolded aorta, it is not possible to entirely rule out that part of the outlet region of the present invention will touch the vascular wall. However, this is prevented to the greatest possible extent by the presently disclosed configuration of the outlet region.

As a further technical advantage, due to the outer shape of the stent structure of the present invention, a stable valve geometry is defined that changes relatively little in the provided diameter range of the annulus.

Furthermore, avoiding buckling in the stent structure in the longitudinal direction (“infolding”) is a critical advantage, both when re-sheathing the stent structure in a catheter capsule and when the stent structure is implanted in highly irregularly shaped annuli and annuli with major calcifications.

Likewise, advantageously, the requirement for a shortened implanted stent with the simultaneous requirement for sufficient length of the stent structure for the implantation process is addressed by the present configurations of the stent structure to permit precise positioning during implantation. This is realized in that, due to the specific distribution of different strut lengths and widths, during the implantation process different regions in the stent structure of the present invention shorten in the desired manner or intentionally do not shorten.

Additional advantages of the stent structure of the present invention to be cited are as follows:

The optimized longitudinal height of the stent structure, which is realized by an optimized design of the struts in the stent structure, makes possible, in particular, multiple repositionings of the stent structure during an implantation. Surprisingly, it was possible to optimize the elongation behaviour using intentional variation and distribution of different strut lengths and slightly increased strut widths.

Another critical advantage of the invention is increased sealing of the stent structure against the surrounding anatomy, which prevents paravalvular leaks. The sealing tightness of the prosthesis of the present invention, that is, of the stent structure and valve arrangement, is attained using a specially configured cell structure in the inlet region that nevertheless has sufficient build-up of radial force. To connect the finely netted region of the inlet to the coarsely netted outlet, according to the invention a transition zone is defined between inlet region and outlet region of the stent structure and provides free and generous access to the coronary arteries. Correspondingly, the cells in the outlet region are likewise configured with sufficient size.

Consequently, the stent structure of the present invention also ensures access to the prosthesis with a conventional catheter for diagnostic purposes or further implantation of another prosthetic heart valve (valve-in-valve principle).

With the aforesaid advantages combined, the present invention provides a prosthetic heart valve that includes a stent structure having optimized radial force, improved access to the coronary arteries, and increased stability against infolding when the stent structure is repositioned. These technical advantages are made possible by an intentional arrangement of cells, their intentional definition and distribution of strut lengths and strut widths, and the described outer shaping of the stent structure.

As mentioned herein, the prosthetic heart valve of the present invention may assume a number of different configurations, such as, e.g., a bioprosthetic heart valve having tissue leaflets made of pericardium, a valve having polymer, metal, or tissue leaflets, and may be specially configured for replacing one of the four valves in the human heart, aortic valve replacement being most preferred.

The valve arrangement including a valve structure may assume a number of shapes and may be formed, for example, from one or a plurality of biocompatible plastics, synthetic polymers, autograft tissue, homograft tissue, xenograft tissue, or one or a plurality of other suitable materials.

In some embodiments, the valve structure may be formed, for example, from bovine tissue, porcine tissue, equine tissue, ovine tissue, and/or other suitable animal tissue.

In one embodiment, the valve structure may be formed, for example, from kangaroo tissue.

In one embodiment, the valve structure may be formed, for example, from a suitable 3D-printed material.

In some embodiments, the valve structure may be formed, for example, from heart valve tissue, pericardial tissue, and/or other suitable tissue, porcine pericardial tissue being most preferred.

In some embodiments, the valve arrangement may include one or a plurality of valve leaflets. Thus, for example, the valve arrangement may be in the form of a bovine or porcine tricuspid pericardial valve, a bicuspid valve, or another suitable valve.

In some designs, the valve arrangement may include two or three valve leaflets that are joined at enlarged lateral end regions to be fixed to commissure posts, wherein the unbound edges form coaptation edges of the valve arrangement, wherein one commissure post may include, for example, at least three holes arranged one after the other in longitudinal direction, at least two elongated holes arranged one next to the other in circumferential direction, a plurality of sideway notches, curved material surrounding one hole and/or a plurality of hooks. All aforementioned elements of the commissure post are located in the circumferential plane of the stent structure.

In one preferred embodiment, the prosthetic heart valve of the present invention may be configured for replacing or repairing an aortic valve (e.g. with respect to size and shape). Alternatively, other shapes are provided that follows the specific anatomy of the valve to be repaired (e.g., the prosthetic heart valve of the present invention may be alternatively shaped and/or dimensioned for replacing a native mitral, pulmonary, or tricuspid valve).

The prosthetic heart valve of the present invention may be delivered in different manners to the target heart valve using various transluminal delivery instruments as are known in the prior art. In general, the prosthetic heart valve is compressed in this process and held in an outer delivery device or capsule and then in this compressed state is advanced to the target location before the prosthesis is ultimately released (e.g. by retracting the capsule).

Correspondingly, another aspect of the invention refers to a method for using the prosthetic heart valve, which method is characterized in that it includes the following steps:
transporting a prosthetic heart valve of the invention to the native heart valve, wherein the step of transporting the prosthetic heart valve includes holding the stent structure in the compressed state inside a delivery device;
supplying the prosthetic heart valve, including the stent structure that automatically expands in the direction of the natural state, to the native heart valve from the delivery device; and,
aligning the inlet region in a desired anatomical position of the native heart valve. In one embodiment the desired anatomical position is in the annulus plane.

The invention further is directed to a method for manufacturing the above described prosthetic heart valve, including at least the following steps
cutting the mesh structure of the stent structure, wherein the mesh structure has an essentially tubular shape and that furthermore defines a circumference with contours from a tubular body, wherein the contours define the proximal inlet region and the distal outlet region, and wherein the mesh structure includes a plurality of closed cells that in the longitudinal direction of the prosthetic heart valve have varying cell sizes and cell configurations, and thus includes a plurality of cell patterns that vary in size between the proximal inlet region and the distal outlet region, wherein the mesh structure forms meander rows of struts and nodes where struts merge,
expanding the mesh structure to its final outer shape and annealing the expanded mesh structure, wherein during expansion the nodes located at the distal end of the first meander row is fixed in circumferential direction in the expanding tool,
suturing or gluing valve leaflets at least to an inner skirt element and suturing or gluing the inner skirt element itself to the stent structure forming the valve arrangement that is arranged inside the lumen of the stent structure. With above method, in particular the fixing of nodes located at the distal end of the first meander row during expansion in the expanding tool in circumferential direction, it is avoided that cells in the inflow region are deformed irregularly but form regular cells. Thereby, longitudinal buckling of the stent structure during implantation or one or more re-sheathing processes of a prosthesis is avoided.

Although the present disclosure has been described with reference to various embodiments and preferred embodiments thereof, the person skilled in the art will discern that additional changes may be made in shape and details without departing from the original scope of the present disclosure.

Correspondingly, in the context of the present invention all details and embodiments of the prosthetic heart valve may be combined as desired and may be used with the disclosed prosthetic heart valve and with the method for its use. All details and embodiments of the prosthetic heart valve and of the method for its use may likewise be combined as desired and used for any other vascular prosthesis in any desired combination.

Provided there is no indication to the contrary, the general tolerance for the strut width is +/−0.015 mm and for the wall thickness is +/−0.020 mm. The following figures are schematic drawings showing different embodiments of prosthetic heart valves or sections of them.

FIG. 1: Schematic depiction of the outer contours of one embodiment of the prosthetic heart valve of the present invention. Z1 designates a conical-convex inflow region (anulus zone) that is defined by a first diameter DI and a second diameter D2 (“Belly nadir” diameter), D1 being greater than D2, and the outer surface area of this Z1 region being characterized in that it is curved outward in the longitudinal direction (convex or double curved). Connected to the region Z1 is a cylindrical and thus linear outflow zone Z3 (outflow region) that is characterized in that the first diameter D3 remains the same in the entire zone Z3. This leads to a so-called “linear outflow” for the vascular implant. Finally, a connector may be attached to the distal end of the outflow zone Z3. In one embodiment the connector zone may include six connectors. In another embodiment the connectors, optionally six connectors, may be single-stranded and may furthermore have atraumatic tip elements (not shown). The sections inflow region Z1 and outflow region Z3 are arranged along a longitudinal direction of the heart valve, wherein the diameters D1, D2 and D3 extend perpendicular to the longitudinal direction.

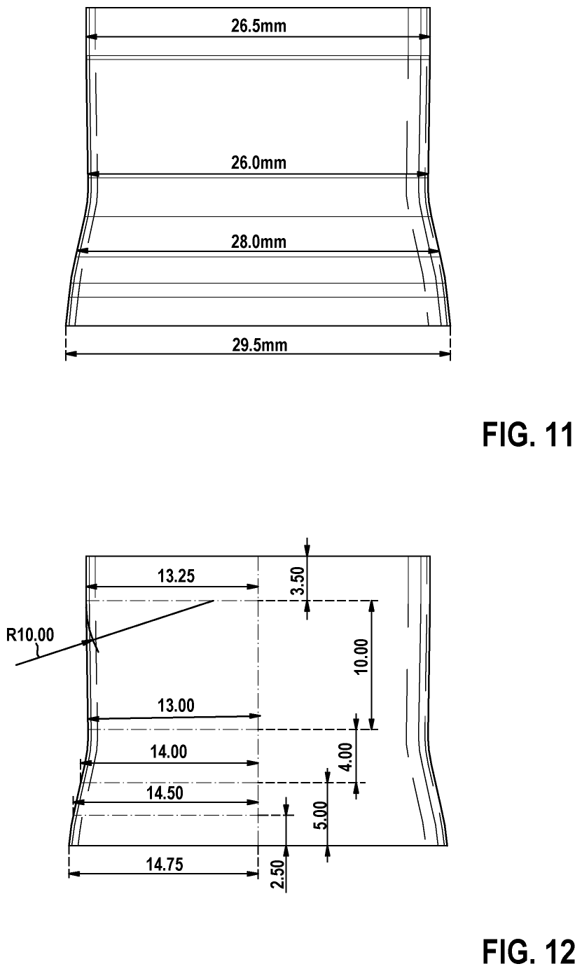

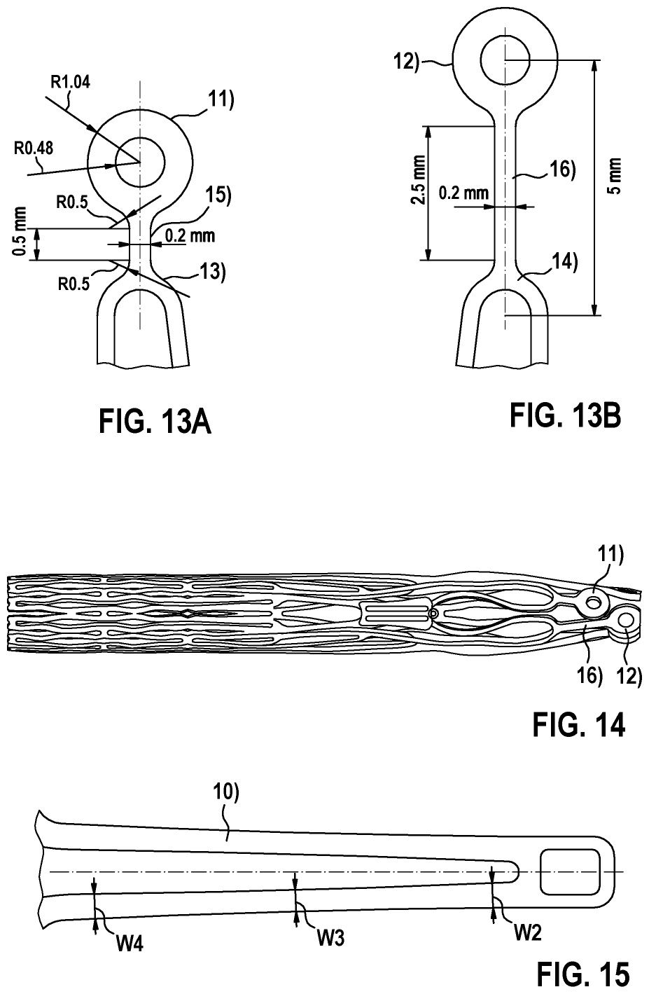

FIGS. 2, 11 and 12: Depiction of a stent structure in a side view according to one embodiment of the prosthetic heart valve of the present invention based on the characteristics of FIG. 1. Z1 designates a conical-convex inflow region (annulus zone) that is defined by a first diameter DI and a second diameter D2 (“Belly nadir” diameter), D1 being larger than D2 and the outer surface area of this Z1 region being characterized in that it is curved outwardly in the longitudinal direction (convex or double curved). The diameter D3 (so-called “Attachment” diameter) is further characterized in that the second diameter D2 is smaller than the third diameter D3. Attached to zone Z1 is a cylindrical and thus linear outflow zone Z3 (outflow region) that is characterized in that the diameter D3 remains the same in the entire zone Z3. This leads to a so-called “linear outflow” for the prosthetic heart valve. The connectors, for example six connectors 3), are illustrated at the distal end of the outflow zone Z3. The structure of the connectors 3) is further explained below with respect to FIG. 13. FIG. 2 shows that the cells 1) in the inflow region Z1 are smaller than the cells 2a) of the outflow region Z3. In particular, the outflow region Z3 has six greater cells 2a) and three smaller cells 2b), wherein a distal section of each cell 2a), 2b) belongs to the outflow region Z3 or forms the outflow region Z3. Further, the stent structure includes three commissure post 4) arranged between the inflow region Z1 and the outflow region Z3 at a connection area of two greater cells 2a). Further the height H of the stent structure (including the connectors 3)) along its longitudinal direction is, for example, 33 mm. As depicted in FIG. 11 the diameter D1 may be, for example, 29.5 mm, the diameter D2, for example, 26 mm and the diameter D3, for example 26.5 mm. Further dimensions of the prosthetic heart valve or the stent structure may be derived from FIG. 12. The distance of the second diameter D2 (“belly nadir”) may be chosen, for example, as 9 mm. The height of the valve arrangement is, for example, 13.5 mm into longitudinal direction when attached to the stent structure (not shown).

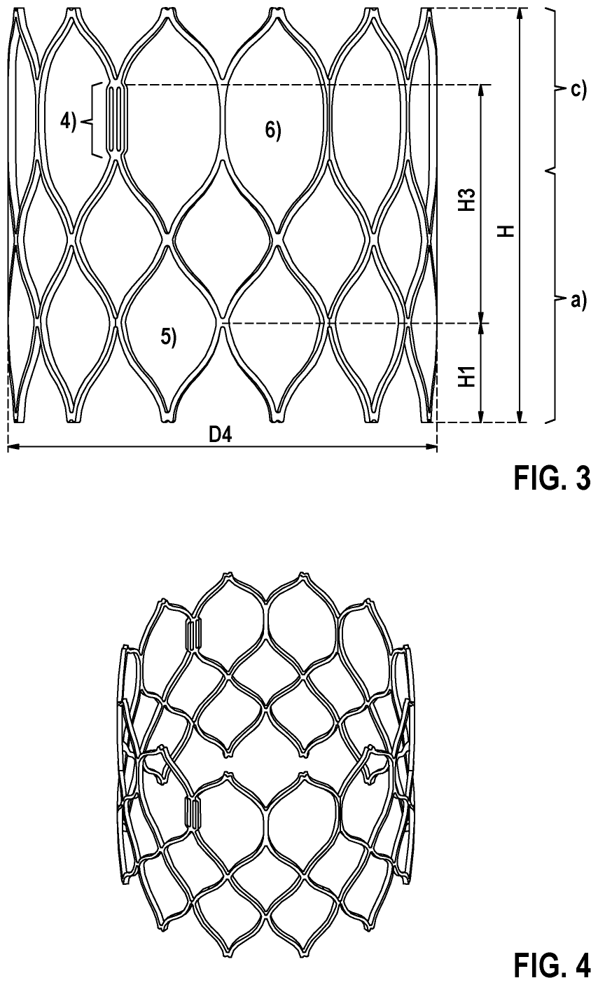

FIGS. 3 and 4: FIGS. 3 and 4 depict a stent structure according to a second embodiment of the prosthetic heart valve of the present invention, wherein FIG. 3 shows a side view. FIG. 4 shows a perspective view of the stent structure of FIG. 3 from the side. The structure may include connectors as described for the embodiment of FIG. 2. The embodiment illustrated is characterized by a stent structure that has a certain number of cells (e.g. 12 cells, see reference number 5)) in the circumferential direction in the inflow region a), wherein the aforesaid number of cells is divisible by 3, and there is a further number of cells in the circumferential direction in the outflow region c) (see reference number 6)), also divisible by 3, both numbers are identical. Both regions a) and c) are connected to one another and the outflow region c) in this embodiment includes the largest cells 6) in the stent structure for free access to the coronary arteries. The cells 6) of the outflow region c) have a honey-comb (hexagonal) shape. The height H1 of the first meander row located at the furthest proximal end is, for example, 6 mm. The height H3 from the distal end of the first meander row to the distal end of the commissure post 4) with two slits is, for example, 14.4 mm. The main strut width may be, for example, 0.35 mm and the wall thickness, which is constant over the stent structure, may be, for example, 0.465 mm. The height H of the stent structure (please note there are no connectors) is, for example, 25 mm.

The stent structure includes three commissure posts 4) between the inflow region a) and the outflow region c), in particular at struts oriented in longitudinal direction of the honey-comb shaped cells 6).

The stent structure includes a linear inflow region a) and a linear outflow region c), both having the same diameter D4 of, for example, 29 mm.

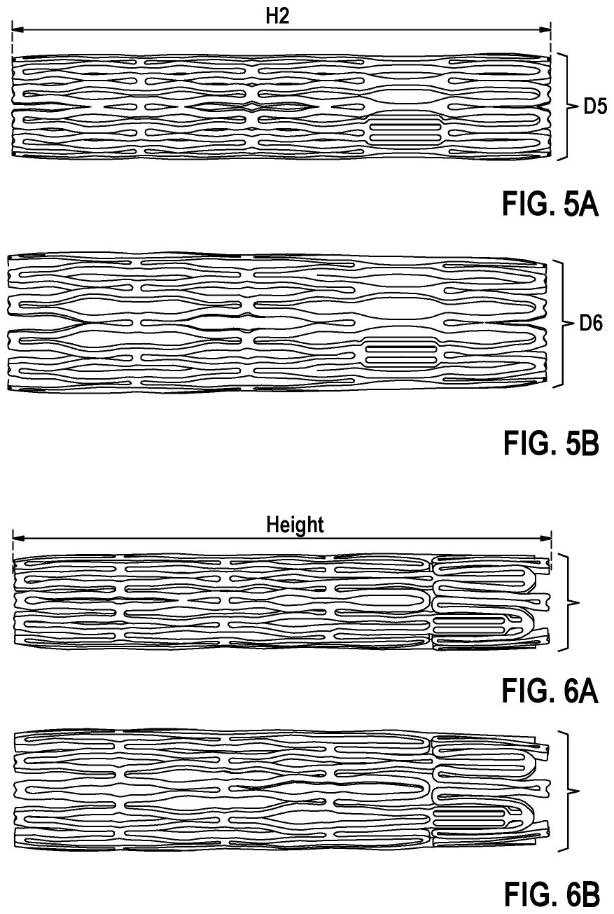

FIGS. 5A and 5B show crimped states of the stent structure, wherein FIG. 5A shows the stent structure crimped by a crimp tool, which radial force is active, to a diameter D5 of 4.9 mm and a height H2 of 29.36 mm. FIG. 5B shows the stent structure when the crimp tool is not active (i.e. no exterior forces act on the stent structure), so that an elastic recoil occurs to a diameter D6 which is greater than D5. For this state the height H2 is, for example 29.33 mm which is almost equal to H2 for the stent structure when the radial face is active.

In the context of the invention, the stent structure having an inflow region and an outflow region as described in the foregoing for FIG. 3 may furthermore be designed such that the foreshortening of the inflow region and of the outflow region may be influenced independently of one another using the specific embodiment of the stent elements (i.e., using strut width and strut length) so that the length of the stent in these regions may be adjusted during and following implantation to cause a desired foreshortening compensation in these regions, independently of one another, and/or to minimize undesired foreshortening compensation.

Referring to FIG. 3, in one embodiment this exemplary stent structure is further characterized in that three closed zig-zag rows (so-called zig-zags or meanders) in the inflow region a) are provided with a strut length adjusted to the entry diameter. The height H1 of the first zig-zag row is greater than the length of the second two zig-zag rows.

FIGS. 6A and 6B show crimped states of the stent structure, wherein FIG. 6A shows the stent structure crimped by a crimp tool, which radial force is active, to a diameter D5 of 4.9 mm and a height H2 of 29.38 mm. FIG. 6B shows the stent structure when the crimp tool is not active (i.e. no exterior forces act on the stent structure), so that an elastic recoil occurs to a diameter D6 which is greater than D5. For this state the height H2 is, for example 29.33 mm which is almost equal to H2 for the stent structure when the radial face is active.

In the context of the invention, the stent structure having an inflow region and an outflow region as described in the foregoing for FIG. 3 may furthermore be designed such that the foreshortening of the inflow region and of the outflow region may be influenced independently of one another using the specific embodiment of the stent elements (i.e., using strut width and strut length) so that the length of the stent in these regions may be adjusted during and following implantation to cause a desired foreshortening compensation in these regions, independently of one another, and/or to minimize undesired foreshortening compensation.

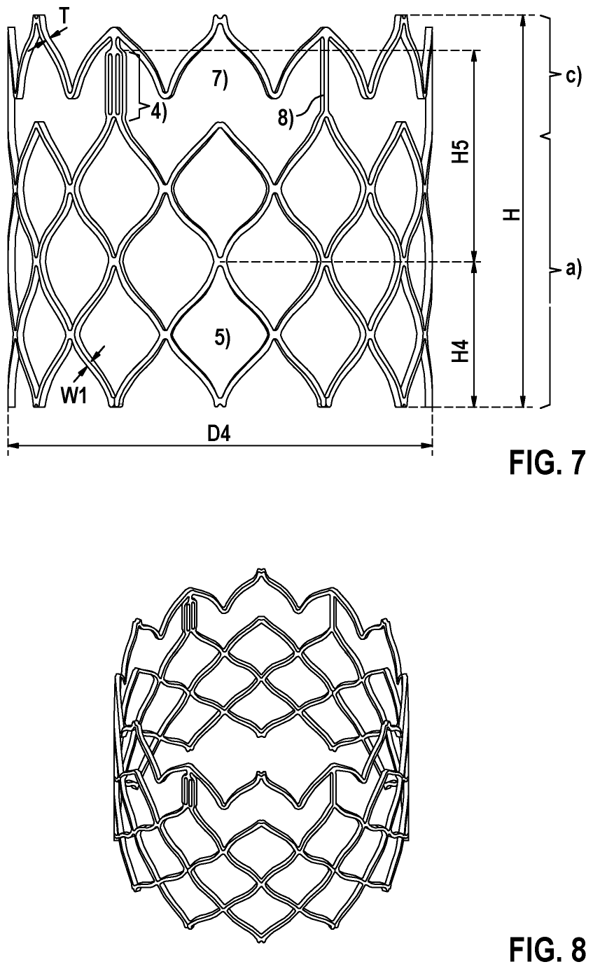

FIGS. 7, 8 and 9: FIGS. 7 to 9 depict a stent structure according to a third embodiment of the prosthetic heart valve of the present invention, wherein FIGS. 7 and 9 show side views. FIG. 8 shows a perspective view of the stent structure of FIG. 7 from the side. The embodiment illustrated is characterized by a stent structure that has a certain number of cells (e.g. 12 cells, see reference number 5)) in the circumferential direction in the inflow region a), wherein the aforesaid number of cells is divisible by 3, and there is a further number of cells in the circumferential direction in the outflow region c) (see reference number 7)), also divisible by 3, both numbers are identical. Both regions a) and c) are connected to one another and the outflow region c) in this embodiment includes the largest cells in the stent structure 3) for free access to the coronary arteries. The cells 7) of the outflow region c) have a W-shape which is caused by two parallel meander rows at the distal end of the stent structure which are connected at their opposite apices by six struts 8) extending in longitudinal direction. The height H4 of the first cell row located at the furthest proximal end is, for example, 9.1 mm. The height H5 from the distal end of the first cell row to the distal end of the commissure post 4) with two slits is, for example, 13.4 mm. The main strut width W1 (see FIG. 7) may be, for example, 0.35 mm and the wall thickness T (see FIG. 7), which is constant over the stent structure, may be, for example, 0.465 mm. The height H of the stent structure (please note there are no connectors) is, for example, 25 mm.

The stent structure includes three commissure posts 4) between the inflow region a) and the outflow region c), in particular, at struts 8) oriented in longitudinal direction of W-shaped cells 6).

The stent structure includes a linear inflow region a) and a linear outflow region c), both having the same diameter D4 of, for example, 29 mm.

Referring to FIG. 7, in one embodiment this exemplary stent structure is further characterized in that three closed zig-zag rows (so-called zig-zags or meanders) in the inflow region a) are provided with a strut length adjusted to the entry diameter. The height H1 of the first zig-zag row is greater than the length of the second two zig-zag rows.

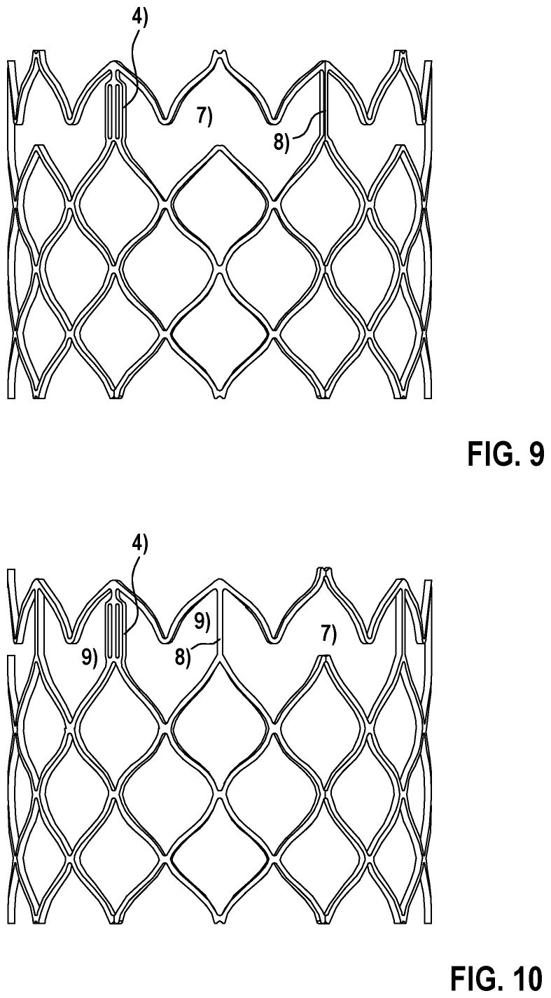

FIG. 10 corresponds to the embodiment shown in FIGS. 7 to 9 with a different arrangement of the longitudinal struts 8) of the outflow region c). The longitudinal struts 8) are repositioned and added such that they extend from the opposite apices of the opposite meander structures adjacent to the longitudinal strut including the commissure post 4). Thereby, the outflow region c) is stabilized to improve the crimping behaviour. Accordingly, the outflow region now includes W-shaped cells 7) and V-shaped cells 9).

FIGS. 11 and 12: depicts by way of example an exemplary outer shape of the stent structure of the present invention according to FIG. 1 in the expanded state having exemplary dimensions and angles in the context of the invention [in mm].

FIGS. 13A, 13B and 14: show schematic details of a shifted connectors 3) of the embodiment of FIG. 2 with some corresponding dimensions [in mm] and angles. The stent structure includes three first connector elements 11) and three second connector elements 12) at its distal end, each of the first connector element 11) and the second connector element 12) is connected to a respective apical node 13), 14), wherein a first strut 15) connecting the first connector element 11) to a first node 13) has a different length compared to a second strut 16) connecting the second connector element 12) to a second node 14). In this embodiment the first strut 15) is shorter than the second strut 16). The first node 13) and the second node 14) are located adjacent along the circumferential direction of the stent structure (see FIG. 2). In this embodiment, the connector elements 11), 12) are shifted against each other so that the diameter of the stent structure in the crimped state is less since the first connector element 11) is located adjacent the second strut 16) of the adjacent second connector element 12). The crimped state is depicted in FIG. 14. FIG. 13A shows one of the first connector elements 11 and including an eyelet which is connected to the first strut 15. The first strut has a length of 0.5 mm (might be also between 0.2 mm and 1.5 mm) and a width of 0.2 mm (might be also between 0.1 mm and 0.5 mm). The eyelet of the first connector element has an outer diameter which is more than 0.2 mm, preferably between 0.4 mm and 2.5 mm. FIG. 13B shows one of the second connector elements 12 and including an eyelet which is connected to the second strut 16. The second strut 16 has a length of 2.5 mm (might be also between 2.0 mm and 5 mm) and a width of 0.2 mm (might be also between 0.1 mm and 0.5 mm). The eyelet of the second connector element has an outer diameter which is more than 0.2 mm, preferably between 0.4 mm and 2.5 mm.

FIG. 15: Referring to FIG. 15, FIG. 15 provides a detailed excerpt from the embodiment of FIG. 2 (see proximal end of the stent structure) of the stent structure that is characterized in that the struts 10) have a strut width that may vary along the struts, so that the width at one positions (for example W3) along the strut is minimal, whereas the strut width at the two positions (W2, W4) is greater than at position W3 but the same as or smaller than the strut width at the nodes (so-called “waist”).

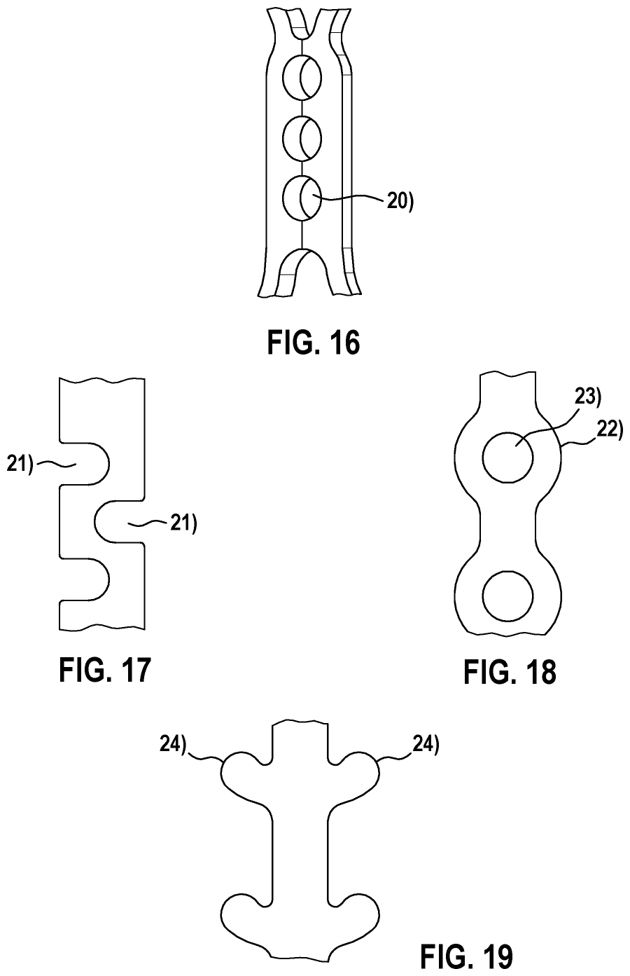

FIGS. 16 to 19: provides various schematic details of a mesh structure for the stent structure of the present invention showing different commissure posts for attachment of the valve arrangement at the stent structure. FIG. 16 shows an arrangement with three circular holes 20) arranged one after the other in longitudinal direction of the stent structure, FIG. 17 a strut with a plurality of sideway notches 21), alternately located at the opposite side edges of the strut and arranged one after the other in longitudinal direction, FIG. 18 curved material 22) of the strut surrounding one hole 23), and FIG. 19 a plurality of hooks 24) formed by projections in radial direction from a longitudinal strut.

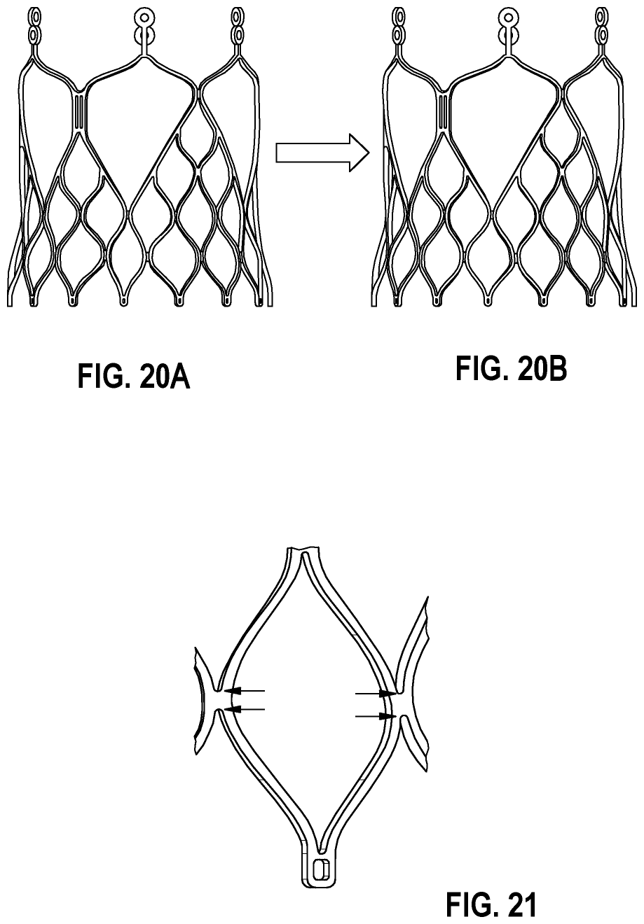

FIGS. 20A, 20B and 21: FIG. 20A and 20B show one step of the manufacturing method of the stent structure of the embodiment of FIG. 2. During manufacturing the stent structure is expanded to its final outer shape and annealed. In one embodiment, if the stent structure will be expanded without any fixing, the cells of the most proximal row of the inflow region Z1 are deformed irregularly (see FIG. 20A). FIG. 20B shows the state after expansion where the nodes at the distal end of the first meander structure (see double arrows in FIG. 21 which is a detail of FIG. 20A/B) were fixed in circumference direction in the expanding tool. The cells in the expanded state as shown in the right drawing are regular and show less buckling problems.

Referring to the aforesaid disclosure, the present invention further includes the following embodiments:

A. A prosthetic heart valve, including:
a stent structure that is configured to expand from a compressed state for transluminal delivery to a natural expanded state and having an overall height (H), the stent structure including a mesh structure that has an essentially tubular shape and that furthermore defines a circumference with contours, wherein the contours define a proximal inlet region (Z1, a) and a distal outlet region (Z3, c), wherein the inlet region (Z1, a) and outlet region (Z3, c) are directly connected to one another, and wherein the mesh structure includes a plurality of closed cells (1, 2a, 2b, 4 to 7 and 9) that in a longitudinal direction of the prosthetic heart valve have varying cell sizes (H4) and cell configurations, and thus includes a plurality of cell patterns (1, 2a, 2b, 4 to 7 and 9) that vary in size between the proximal inlet region (Z1, a) and the distal outlet region (Z3, c), and
a valve arrangement that is arranged inside a lumen of the stent structure, characterized in that the overall height (H) of the stent structure is low, preferably less than 45 mm, further preferably less than 40 mm, more preferably less than 35 mm.

With the context of the invention the expression “overall height (H)” denotes in case of at least one connector element being present on the stent structure that the connector length is included in overall height (H). If, however, no any connector element is present on the stent structure, this means that only the stent height itself is accounting for overall height (H) measurements.

B. A prosthetic heart valve, including:
a stent structure that is configured to expand from a compressed state for transluminal delivery to a natural expanded state, the stent structure including a mesh structure that has an essentially tubular shape and that furthermore defines a circumference with contours, wherein the contours define a proximal inlet region (Z1, a) and a distal outlet region (Z3, c), wherein the inlet region (Z1, a) and outlet region (Z3, c) are directly connected to one another, and wherein the mesh structure includes a plurality of closed cells (1, 2a, 2b, 4 to 7 and 9) that in a longitudinal direction of the prosthetic heart valve have varying cell sizes and cell configurations, and thus includes a plurality of cell patterns (1, 2a, 2b, 4 to 7 and 9) that vary in size (H4) between the proximal inlet region (Z1, a) and the distal outlet region (Z3, c), and
a valve arrangement that is arranged inside a lumen of the stent structure, characterized in that
   the cells in the inlet region (1) are formed and arranged (5) for defining in the stent structure
a conical-convex outer shape across the entire circumference of the inlet region (Z1; i.e. a so-called “conical-convex inlet region” or “conical-convex inflow region”) or
a linear cylindrical outer shape across the entire circumference of the inlet region (a; i.e. a so-called “linear cylindrical inlet region” or “linear cylindrical inflow region”), and
   the cells in the outlet region (2) are shaped and arranged for defining in the stent structure a linear cylindrical outer shape across the entire circumference of the outlet region (Z3, c; i.e. a so-called “linear cylindrical outlet region” or “linear cylindrical outflow region”).

C. The prosthetic heart valve according to embodiment A or B, wherein the cells (1,5) of the inlet region (1) and the outlet region (2) are configured differently from one another to always build up, when the stent structure is in the expanded state, a higher maximum radial force in the inlet region (Z1) in direct comparison to a lower maximum radial force in the outlet region (Z3).

D. The prosthetic heart valve according to any of embodiments A to C, wherein the valve arrangement includes a plurality of valve leaflets that are arranged for defining an entry side and an exit side in the prosthetic heart valve.

E. The prosthetic heart valve according to any of embodiments A to D, wherein the valve arrangement is having two or more valve leaflets, for example three valve leaflets, and includes an inner skirt element on the inside of the stent structure, wherein said valve leaflets are sutured or glued at least to the inner skirt element and the inner skirt element is itself sutured or glued to the stent structure.

F. The prosthetic heart valve according to any one of embodiments A to E, wherein the stent structure furthermore includes an outer skirt element on the outside of the stent structure, wherein the outer skirt element is sutured or glued at least to the stent structure.

G. The prosthetic heart valve according to any one of embodiments A to F, wherein in the expanded state
the inlet region is a conical-convex inlet region (Z1) defining a first diameter (D1) or
the inlet region is a linear cylindrical inlet region (a),
   and the outlet region is a linear cylindrical outlet region (Z3) defining a second diameter (D3), further characterized in that the first diameter (D1) is larger than the second diameter (D3).

H. The prosthetic heart valve according to embodiment G, wherein the inlet region (Z1), when the stent structure is in the expanded state and forms the conical-convex inlet region, defines a third diameter (D2) that is smaller than said first diameter (D1) and second diameter (D3).

I. The prosthetic heart valve according to any one of embodiments A to H, further characterized in that the conical-convex inlet region expands proximally throughout the inlet region (Z1).

J. The prosthetic heart valve according to any one of embodiments A to H, each of the closed cells (1, 2, 4 to 7 and 9) includes a plurality of struts (8) connected to one another, and each strut itself includes a plurality of segments connected to one another, and wherein furthermore a geometry of at least one of the struts and/or of at least one of the segments of the closed cells in the inlet region (Z1, a) is smaller than a corresponding geometry of a corresponding strut and/or a corresponding segment in the closed cells of the outlet region (Z3, c).

K. The prosthetic heart valve according to embodiment J, wherein the geometry is a length of a strut (H1) and/or of a segment of the strut.

L. The prosthetic heart valve according to embodiment J or k, wherein the geometry is a width (W1 to W4) of a strut and/or a segment of the strut.

M. The prosthetic heart valve according to any one of embodiments A to L, further characterized in that the outlet region (Z3) at the distal end includes one or more connector elements (11, 12), for example, two, three or six connector elements.

N. The prosthetic heart valve according to any of the embodiments A to M, wherein the outlet region (Z3) at its distal end or a section distal from the outlet region includes at least one first connector element (11) and at least one second connector element (12), each of the first connector element and the second connector element is connected to a respective first apical node (13) or second apical node (14), wherein a first strut (15) connecting the first connector element (11) to the first node (13) has a different length compared to a second strut (16) connecting the second connector element (12) to the second node (14), wherein the first node (13) and the second node (14) are arranged sequentially along the circumferential direction of the stent structure.

O. The prosthetic heart valve according to embodiment M or N, wherein at least one of the connector elements (including the first and the second connector elements 11, 12)) includes an eyelet.

P. The prosthetic heart valve according to any one of embodiments A to O, further characterized in that the inlet region includes one or more cell rows.

Q. The prosthetic heart valve according to any one of embodiments A to P, wherein the inlet region (Z1, a) includes one or more cell rows to define a first band of closed cells that extend about an entirety of the circumference of the inlet region, wherein the closed cells of the first band (1) are spaced equidistant from one another along the circumference, and further wherein the first band is configured to always build up the highest radial force along the circumference when the stent structured is in the expanded state in direct comparison to the radial force of the rest of the stent structure.

R. The prosthetic heart valve according to any one of embodiments A to Q, wherein the inlet region (Z1, a) includes one or more cell rows including 12, 15, or 18 cells, wherein the cells of one row may, for example, be formed as a rhombohedral cells (1, 2b, 5), honey-comb shaped cells (6), V-shaped cells, W-shaped cells or N-shaped cells.

S. The prosthetic heart valve according to any one of embodiments A to R, wherein the inlet region (Z1, a) includes at least three meander rows.

T. The prosthetic heart valve according to any one of embodiments A to S, further characterized in that the proximal apical tips of the cells in the inlet region, in direction of the inflow, have a nutcracker shape or an eyelet for fixing sutures of the valve arrangement.

U. The prosthetic heart valve according to any one of embodiments A to T, wherein the outlet region (Z3) has one row of closed cells (2a, 2b, 6) including 3 to 9 cells.

V. The prosthetic heart valve according to any of the embodiments A to R, wherein the outlet region (Z3, c) consists of one meander row or two meander rows.

W. The prosthetic heart valve according to any of the embodiments A to V, wherein the inlet region (Z1, a) consists of three or four meander rows and the outlet region (Z3, c) consists of one meander row, wherein the one meander row of the outlet region (Z3) runs parallel compared with the most distal meander row of the inlet region (Z1) and are connected with struts extending in the longitudinal direction.

X. The prosthetic heart valve according to any one of embodiments A to W, further characterized in that, for fixing the valve arrangement, at least one commissure post (4) is arranged between the inlet region (Z1) and the outlet region (Z3) or at the proximal end of the outlet region of the mesh structure in the stent structure, wherein one commissure post includes, for example, at least two, preferably at least three holes (20) arranged one after the other in longitudinal direction, at least two elongated holes arranged one next to the other in circumferential direction, a plurality of sideway notches (21), curved material (22) surrounding at least one hole (23) and/or a plurality of hooks (24).

Y. The prosthetic heart valve according to any of embodiments D to X, wherein the valve leaflets are formed from a material selected from the group consisting of biological material, cellulose, porcine, bovine, equine, or other mammalian pericardial tissue, synthetic material, or polymeric material.

Z. The prosthetic heart valve according to any one of embodiments D to y, wherein the valve leaflets being deployable supra-annularly from the aortic annulus of a patient when the prosthetic heart valve is advanced inside the aortic valve of a patient and the stent structure is in the expanded state.

AA. The prosthetic heart valve according to any one of embodiments E to Z, wherein the inner skirt is being formed from a material selected from the group consisting of biological material, cellulose, porcine, bovine, equine, or other mammalian pericardial tissue, synthetic material, or polymeric material.

BB. The prosthetic heart valve according to any of embodiments F to AA, wherein the outer skirt is being formed from a material selected from the group consisting of biological material, cellulose, porcine, bovine, equine, or other mammalian pericardial tissue, synthetic material, or polymeric material.

CC. The prosthetic heart valve according to any of embodiments A to BB, wherein the strut width (W1 to W4) of the stent structure is at least 0.10, preferably at least 0.15, more preferably at least 0.20, most preferred at least 0.25 mm.

DD. The prosthetic heart valve according to any one of embodiments A to CC, wherein the stent structure is being configured for holding the native heart valve of a patient continuously open when in the expanded state.

EE. The prosthetic heart valve according to any one of the embodiments A to DD, wherein the prosthetic heart valve is being configured as a replacement for a native aortic valve.

FF. A prosthetic heart valve, including:
a stent structure that is configured to expand from a compressed state for transluminal delivery to a natural expanded state, the stent structure including a mesh structure that has an essentially tubular shape and that furthermore defines a circumference with contours, wherein the contours define a proximal inlet region (Z1) and a distal outlet region (Z3), and wherein the mesh structure includes a plurality of closed cells (1, 2a, 2b) that in a longitudinal direction of the prosthetic heart valve have varying cell sizes and cell configurations, and thus includes a plurality of cell patterns that vary in size between the proximal inlet region (Z1) and the distal outlet region (Z3), and
a valve arrangement that is arranged inside a lumen of the stent structure, characterized in that
   the outlet region (Z3) includes at least one first connector element (11) and one second connector element (12) at its distal end, each of the first connector element and the second connector element is connected to a respective first apical node (13) or a second apical node (14) of the outlet region, wherein a first strut (15) connecting the first connector element (11) to the first node (13) has a different length compared to a second strut (16) connecting the second connector element (12) to the second node (14), wherein the first node (13) and the second node (14) are located sequentially arranged along the circumferential direction of the stent structure.

GG. The prosthetic heart valve according to embodiment FF, wherein at least one of the first connector element (11) and the second connector element (12) includes an eyelet.

HH. A method for treating a patient's native heart valve, the method including the following steps:
transporting a prosthetic heart valve according to embodiments A to GG to the native heart valve, the step of transporting the prosthetic heart valve including holding the stent structure in the compressed state attaching to a delivery device;
supplying the prosthetic heart valve from the delivery device to the native heart valve, including the stent structure that automatically or force-controlled expands in the direction of its natural state; and,
aligning the inlet region in a desired anatomical position of the native heart valve.

II. The method according to embodiment HH, wherein the native heart valve is an aortic valve.

JJ. The method according to embodiment HH or II, wherein the desired anatomical position is in the annulus plane.

KK. A method for manufacturing the prosthetic heart valve according to any of the embodiments A to GG, including at least the following steps
cutting the mesh structure of the stent structure, wherein the mesh structure has an essentially tubular shape and that furthermore defines a circumference with contours from a tubular body, wherein the contours define the proximal inlet region (Z1) and the distal outlet region (Z3), and wherein the mesh structure includes a plurality of closed cells (1, 2, 3) that in the longitudinal direction of the prosthetic heart valve have varying cell sizes (1, 2a, 2b) and cell configurations, and thus includes a plurality of cell patterns (1, 2a, 2b) that vary in size between the proximal inlet region (Z1) and the distal outlet region (Z3), wherein the mesh structure forms meander rows of struts and nodes where struts merge,
expanding the mesh structure to its final outer shape and annealing the expanded mesh structure, wherein during expansion the nodes located at the distal end of the first meander row is fixed in circumferential direction in the expanding tool,
suturing or gluing valve leaflets at least to an inner skirt element and suturing or gluing the inner skirt element itself to the stent structure forming the valve arrangement that is arranged inside the lumen of the stent structure.

1. A prosthetic heart valve, comprising:
a stent structure configured to expand from a compressed state for transluminal delivery to a natural expanded state and having an overall height (H), the stent structure comprising a mesh structure that has an essentially tubular shape and that defines a circumference with contours, wherein the contours define a proximal inlet region and a distal outlet region, wherein the proximal inlet region and the distal outlet region are directly connected to one another, and wherein the mesh structure comprises a plurality of closed cells that in a longitudinal direction of the prosthetic heart valve have varying cell sizes (H4) and cell configurations to form a plurality of cell patterns that vary in size between the proximal inlet region and the distal outlet region, and
a valve arranged inside a lumen of the stent structure, wherein
the stent structure comprises one or more connectors at the distal outlet region and the overall height (H) of the stent structure is less than 45 mm.

2. The prosthetic heart valve according to claim 1, wherein cells of the plurality of closed cells within the proximal inlet region are configured differently than cells of the plurality of closed cells within the distal outlet region.

3. The prosthetic heart valve according to claim 1, wherein the valve comprises one, two or three valve leaflets.

4. The prosthetic heart valve according to claim 1, wherein the valve comprises an inner skirt the inside of the stent structure, wherein said valve is sutured or glued at least to the inner skirt and the inner skirt is itself sutured or glued to the stent structure.

5. The prosthetic heart valve according to claim 1, wherein the stent structure is configured such that in the expanded state
the proximal inlet region is a conical-convex inlet region defining a first diameter or
the proximal inlet region is a linear cylindrical inlet region,
and the distal outlet region is a linear cylindrical outlet region defining a second diameter, wherein the first diameter is larger than the second diameter.

6. The prosthetic heart valve according to claim 5, wherein the proximal inlet region, when the stent structure is in the expanded state and forms the conical-convex inlet region, defines a third diameter that is smaller than said first diameter and second diameter.

7. The prosthetic heart valve according to claim 1, wherein the distal outlet region at its distal end or a section distal from the distal outlet region comprises at least one first connector and one second connector, each of the first connector and the second connector being connected to a respective first apical node or a second apical node of the distal outlet region, wherein a first strut connecting the first connector to the first node has a different length compared to a second strut connecting the second connector to the second node, wherein the first node and the second node are located adjacent along a circumferential direction of the stent structure.

8. The prosthetic heart valve according to claim 1, wherein the proximal inlet region comprises one or more cell rows comprising 12, 15, or 18 cells, formed as one or more of rhombohedral cells, honey-comb shaped cells, V-shaped cells, W-shaped cells or N-shaped cells.

9. The prosthetic heart valve according to claim 1, wherein the proximal inlet region comprises at least three meander rows.

10. The prosthetic heart valve according to claim 1, wherein the distal outlet region comprises one row of closed cells comprising 3 to 9 cells.

11. The prosthetic heart valve according to claim 1, wherein the outlet region consists of one meander row or two meander rows.

12. The prosthetic heart valve according to claim 1, wherein the proximal inlet region consists of three or four meander rows and the distal outlet region consists of one meander row, wherein the one meander row runs parallel to a most distal meander row of the proximal inlet region and the one meander row and the most distal meander row are connected with struts extending in the longitudinal direction.

13. The prosthetic heart valve according to claim 1, comprising a commissure post arranged between the proximal inlet region and the distal outlet region or at a proximal end of the distal outlet region, the commissure post comprising at least three holes arranged one after the other in longitudinal direction, at least two elongated holes arranged one next to the other in circumferential direction, a plurality of sideway notches, curved material surrounding at least one hole and/or a plurality of hooks.

14. The prosthetic heart valve according to claim 1, a strut width of the stent structure is at least 0.10=0.24 mm, preferably at least 0.15 mm, more preferably at least 0.20 mm, most preferred at least 0.25 mm.

15. (canceled)

16. The prosthetic heart valve according to claim 1, comprising one or more connectors as the distal outlet region.

17. The prosthetic heart valve according to claim 16, wherein the one or more connectors each comprise an eyelet.

18. The prosthetic heart valve according to claim 17, comprising at least one first connector and at least one second connector, wherein the first connector is connected to a first node of the stent structure via a first strut and the second connector is connected to a second node of the stent structure via a second strut, and wherein the first strut has a different length than the second strut.

19. The prosthetic heart valve according to claim 18, wherein the first strut has a length which is equal or higher than the sum of the length of the second strut and the length of the at least one second connector.

20. (canceled)

21. The prosthetic heart valve according to claim 18, wherein the overall height (H) of the stent structure including the one or more connectors is more than 27 mm and less than 35-50 mm.

22. (canceled)
